(19) United States
(12) Patent Application Publication
(10) Pub. No.: US 2010/0081895 A1
(43) Pub. Date: Apr. 1, 2010

(54) WIRELESS MEDICAL TELEMETRY SYSTEM AND METHODS USING RADIO FREQUENCY ENERGIZED BIOSENSORS

(76) Inventor: Jason Matthew Zand, Wasington, DC (US)

Correspondence Address: SURGISENSE CORPORATION, 4434 VOLTA PLACE , NW, WASHINGTON, DC, 20007, US

(21) Appl. No.: 12/306,256

(22) PCT Filed: Jun. 21, 2007
(86) PCT No.: PCT/US07/71718

§ 371 Date: Dec. 23, 2008

(51) Int. Cl. A61B 5/00; H04W 4/22

(52) U.S. Cl. 600/309; 455/404.1

(57) ABSTRACT

A system is provided that furnishes physiological or biomechanical parameters from sensors placed upon or within a subject. The system comprises radio frequency energized biosensor (RFEB) devices that are powered by and communicate with an external receiver and monitoring apparatus. One embodiment of the system provides for infant monitoring in a crib utilizing RFEB devices incorporated into a dermal patch. Said patch communicates with a receiver suspended above the crib and alerts of early warning signs of sudden infant death syndrome (SIDS) or other physiologic abnormalities. Another embodiment comprises implantable RFEB devices that relay information related to the condition of an internal tissue, organ, or cavity to an external receiver, monitor, and recorder. Other embodiments include wearable sensors for detecting a subject attempting to get out of bed, dislodge medical equipment, or stray from a given location.

### FIELD OF THE INVENTION

The present invention relates to wireless medical telemetry systems (WMTS), specifically telemetry systems used to gather and relay physiologic parameters through interrogation of radio frequency energized biosensors (RFEB).

### BACKGROUND ART

Wireless medical telemetry is generally used to monitor patient physiological parameters over a distance via radio frequency (RF) communications between a transmitter worn by the patient and a central monitoring station. These devices have the advantage of allowing patient movement without tethering the patient to a bedside monitor with a hard-wired connection.

Traditional WMTS consist of at least one battery powered module worn by the patient for collection and transmission of physiologic parameters. U.S. Pat. No. 6,215,403 discloses a battery powered wireless sensor for monitoring blood oxygen content, and temperature. U.S. Patent Application No. 20020097155 discloses an infant monitor for prevention of sudden infant death syndrome (SIDS); the battery powered sensor straps around the infant's chest, and uses traditional RF communication. The battery powered modules are expensive to produce, susceptible to battery failure, of substantial weight, and produce toxicity hazards. In addition, the traditional patient module consists of a sending unit into which remotely placed physiologic sensor(s) are coupled through hard-wires. This hard-wire coupling creates uncomfortable and potentially hazardous conditions for the patient. Patients become entwined in the web of wires emanating from the sending unit. Furthermore the cost of the patient modules requires that they be reusable. Therefore time consuming decontamination is requisite and adds to patient cost. Sub par cleaning of the modules can lead to transmission of infectious agents. Thus, prior art patient modules are expensive, heavy, cumbersome, and potentially hazardous.

There exists a need for WMTS which collect physiologic/biomechanical data through small, wearable, inexpensive, and disposable transmitters with integrated sensor(s) that are powered by radio frequency energy.

### SUMMARY OF THE INVENTION

An advantage of the present invention is a wireless medical telemetry system which utilizes radio-frequency transmissions to energize and collect physiologic and/or biomechanical data from at least one sensor(s) placed in contact with, or coupled to a subject tissue, organ, appendage, or body of a patient. Sensing modalities include optical sensors, electrical sensors, chemical sensors, mechanical sensors, MEMS sensors, nano sensors, biochemical sensors, acoustic sensors, immunologic sensors, fluidic sensors, ‘lab-on-a-chip’ type sensors, or other types of sensors. Such sensors can be used to detect general health, blood oxygen saturation, blood glucose levels, electrical activity, pulse presence, pulse rate and character, respiratory motion, respiratory rate, temperature, pH, chemical composition, or body motion including acceleration and mechanical shock. The sensors may alternatively be powered by a pre-charged or rechargeable battery, or other power storage device.

Another aspect of the present invention is a system for monitoring a living tissue of a patient's body, comprising a sensor implantable in the patient's body for generating/recording a signal indicative of one or more properties of the tissue; a controller for receiving the signal outside the patient's body; and a communications interface for communicating the signal from the sensor to the controller. A representative application of this system is in the monitoring/recording of a condition at an operative site, or of the condition of a transplanted organ. Other potential applications include but are not limited to the monitoring/recording of intra-cranial, intra-thecal, intra-ocular, intra-otic, intra-nasal, intra-sinusoidal, intra-pharyngeal, intra-laryngeal, intra-esophageal, intra-tracheal, intra-thoracic, intra-bronchial, intra-pericardial, intra-cardiac, intra-vascular, intra-abdominal, intra-gastric, intra-cholecystic, intra-enteric, intra-colonic, intra-rectal, intra-cystic, intra-ureteral, intra-uterine, intra-vaginal, intra-scrotal; intra-cerebral, intra-pulmonic, intra-hepatic, intra-pancreatic, intra-renal, intra-adrenal, intra-lienal, intra-ovarian, intra-testicular, intra-penal, intra-muscular, intra-osseous, and intra-dermal physiologic/biomechanical parameters.

An additional advantage of the present invention is the ability to monitor/record the physiologic/biomechanical parameters of an infant or child. One application is the detection and prevention of SIDS. In this application the RF energized wireless sensors can be affixed to the infant, while the receiver unit can be integrated into a piece of furniture, a bedding material, a toy, a mobile or other accessory. Another application is in the quantification of motion, acceleration, and/or shock for, but not limited to, the following: the detection, and prevention of infant or child abuse, the detection of an injury or fall, and the prevention of travel, or abduction out of preset boundaries.

Another advantage of the present invention is the detection, monitoring, and recording of abnormal physiologic or biomechanical parameters of an infant, child, or adult. Abnormal parameters relate to febrile or convulsive activity, respiratory perturbations, cardiac arrhythmia, and hemodynamic instability.

There is a trend to incorporate RFID tags into hospital supplies for inventory management. An RFID tag reader incorporated into on a patient's wrist band would allow an alarm or other notification to sound if the reader comes in close proximity to sensitive medical equipment that is equipped with an RFID tag. Therefore, a further advantage of the present invention is the detection and resultant prevention of a patient's motion to climb out of bed, and/or dislodge indwelling of external medical equipment including but not limited to endotracheal tubes, IV catheters, thorocostomy tubes, urinary catheters, drainage catheters, naso-/oro-gastric tubes, and percutaneous feeding tubes.

Another aspect of the described invention is the ability to monitor/record a subjects physiology/biomechanical parameters during exercise.

A still further advantage of the present invention is the ability to monitor/record the physiologic/biomechanical parameters of the independent elderly, assisted living/nursing home residents, home care or hospital based patients.

An additional advantage of the present invention is the ability to monitor/record the physiologic/biomechanical parameters of a patient at home for confirmation or quantification of a medical condition. One representative example is the ability to detect and quantify sleep apnea. Another example is the ability to non-invasively measure blood glucose levels in a diabetic patient.

A still further advantage of the present invention is the ability of a superior or medic to assess and monitor the physiologic/biomechanical parameters of a deployed/injured soldier in the battlefield.

Additional advantages of the present invention will become readily apparent to those skilled in this art from the following detailed description, wherein only selected embodiments of the present invention are shown and described, simply by way of illustration of the best mode contemplated for carrying out the present invention. As will be realized, the present invention is capable of other and different embodiments, and its several details are capable of modifications in various obvious respects, all without departing from the invention. Accordingly, the drawings and description are to be regarded as illustrative in nature, and not as restrictive.

### BRIEF DESCRIPTION OF THE DRAWINGS

Reference is made to the attached drawings, wherein elements having the same reference numeral designations represent like elements throughout, and wherein:

FIG. 1 shows a pediatric monitoring system according to an embodiment of the present invention.

FIG. 2 shows an adult monitoring system according to an embodiment of the present invention.

FIG. 3 is a block diagram of a generic wireless medical telemetry system according to an embodiment of the present invention.

FIG. 4 shows an implantable medical telemetry sensor according to an embodiment of the present invention.

FIG. 5 shows an array of sensing elements according to an embodiment of the present invention.

FIG. 6 shows an RF energized biosensor patch according to an embodiment of the present invention.

FIG. 7 shows a block diagram of an oxygenation sensor according to an embodiment of the present invention.

FIG. 8 is a block diagram for an RF energized oxygenation sensor according to an embodiment of the present invention.

FIG. 9a is a graph showing the relationship between light absorption and incident wavelength for varying tissue oxygen saturation.

FIG. 9b is a graph showing an example of light absorption in tissue during de-oxygenation and re-oxygenation.

FIG. 10 is a timing diagram for an RF energized oxygenation sensor according to an embodiment of the present invention.

FIG. 11 is a block diagram of the operation of RF energized oxygenation sensor's operation according to an embodiment of the present invention.

FIG. 12 shows an array of sensing dermal patches for monitoring electrical activity according to an embodiment of the present invention.

FIG. 13 shows a wearable physiological and/or biomechanical sensing patch or armband that communicates with a wearable monitor according to an embodiment of the present invention.

FIG. 14 shows a wearable monitor that detects the proximity of passive RFID tags coupled to medical equipment, beds, cribs, or other devices, and can alert to changes according to an embodiment of the present invention.

FIG. 15 shows a proximity sensing system according to an embodiment of the present invention.

### DESCRIPTION OF THE INVENTION

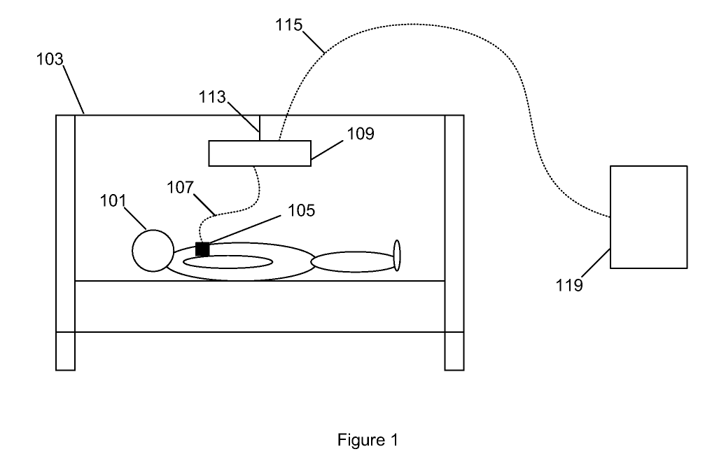

FIG. 1 shows a pediatric monitoring system. One embodiment of the monitoring system is for early detection and prevention of sudden infant death syndrome (SIDS). The infant 101 is in crib 103. Attached to infant 101 is a sensor 105. The sensor 105 can be used to sense one or more physiologic/biomechanical parameters including, but not limited to, general health, systemic blood oxygenation, local tissue oxygenation, pulse (rate, presence, and character), respiration (rate, presence, and character), temperature, electrical activity, moisture, motion, acceleration, and skin stretching or strain. In one embodiment, the sensor 105 is included in a flexible, disposable patch or other pad that is placed on the skin surface and contains no internal power source. In this embodiment, the sensor 105 is powered by techniques known to those skilled in the art of passive/semi-passive radio frequency identification (RFID) by radio frequency (RF) signal 107. RF signal 107 can be emitted by the base antenna 109 or from another external source. Power can be stored in a capacitor, battery, or other storage components. The RF signal energizes the electronics in the sensor 105, which senses the parameter or parameters and then transmits the sensor data over the RF signal 107 to the base antenna 109. In one embodiment, the base antenna and controller 109 is enclosed in a child's toy such as a mobile that is suspended 113 over the crib 103. In other embodiments, base 109 can be a standalone device, integrated directly into a crib, a bed, a bedding material, a stroller, a child's toy such as a stuffed animal, or into a pillow. The base antenna and controller 109 can be communicatively coupled with a base station, baby monitor, or other external device either in the same location or remote location by wires or wireless methods. The connected monitor may be a standard baby monitor with additional feedback, a connection to a nurses' station or monitoring facility, or it may be incorporated into a device such as a pager, pendant, wrist band, mobile phone, portable computer/PDA, telephone dialer, or computer. Monitor information may be transmitted to remote locations through wireless technology including wireless internet, cellular, or other networks, through an internet connection, or through another network.

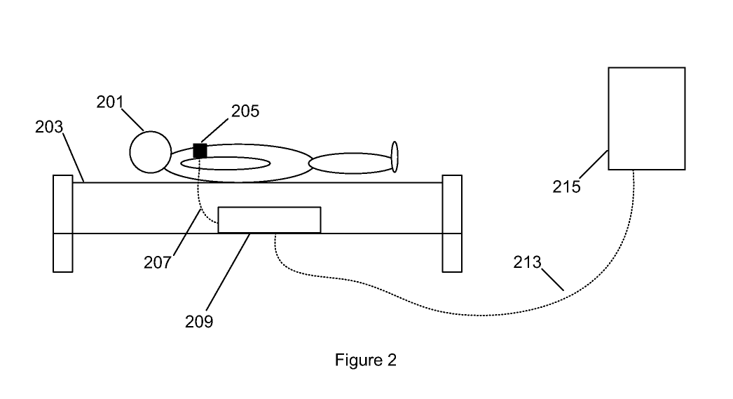

FIG. 2 shows a monitoring system for the elderly, assisted living residents, home care patients, or hospital patients. The functionality is similar to that described in the previous paragraph for FIG. 1. A patient 201 lies on a bed 203. A sensor unit 205 communicates with and is energized by an RF signal 207. A base antenna and controller 209 may be built into the bed, an attachment to the bed, a stand alone device, or a portable, wearable device. The base antenna is communicatively coupled through 213 to an external monitor, controller, phone dialer, alert system, or other external device including those described in the previous paragraph.

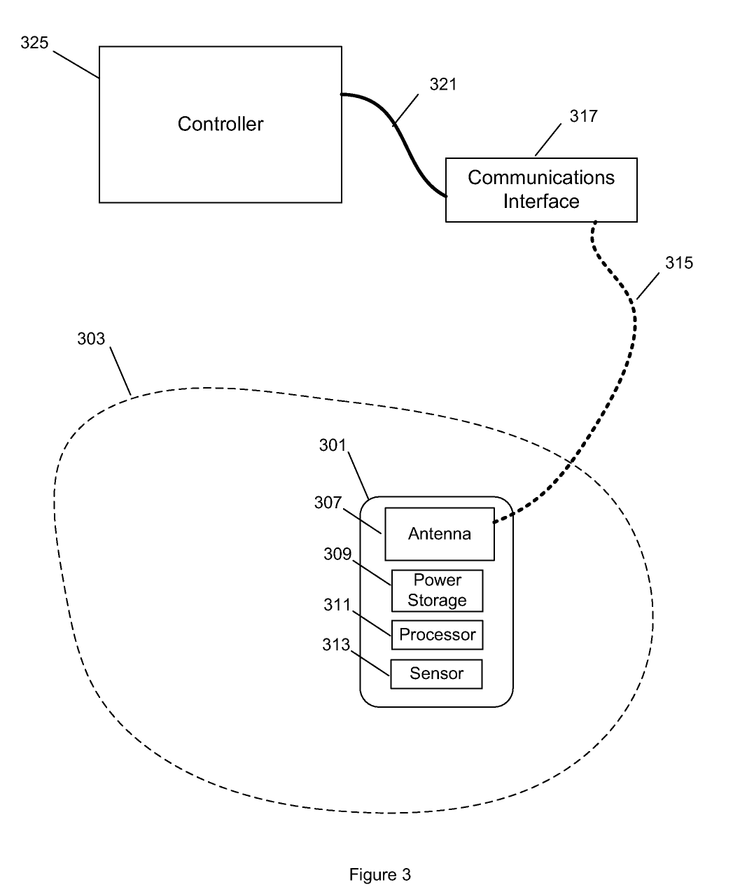

FIG. 3 shows the layout of a generic wireless medical telemetry system with radio-frequency energized sensor. A sensor unit 301 may be embedded into a wearable, self-adhesive, disposable dermal patch/pad. Enclosed in the sensor unit 301 are an antenna 307, a power storage and power regulator system 309, a processor 311, and one or more sensing elements 313. The telemetry sensor device communicates with and is energized by an RF signal 315, which may be emitted from and is received by a communications interface 317. This interface is communicatively coupled via 321 to a controller 325 such as a standard personal computer, stand alone monitoring device, an integrated single unit, or one of the devices described previously. In one embodiment, optical oxygenation and/or pulse sensors are placed on disposable patches that are passively powered and placed on the surface of a patient's skin. Embodiments of these patches can be used in applications including, but not limited to, oxygenation sensing, pulse sensing, temperature sensing, ECG, EMG, chemical sensing including electrolytes or blood sugar with or without a ‘lab-on-a-chip’ type device, motion, acceleration, or mechanical shock monitoring, injury monitoring and assisted triage, exercise and stress test monitoring, infant SIDS monitoring, infant/child abuse surveillance, and elderly, home care, and hospital patient monitoring. The sensors 301 can also be used to detect the proximity of the subject to the communication interface 317.

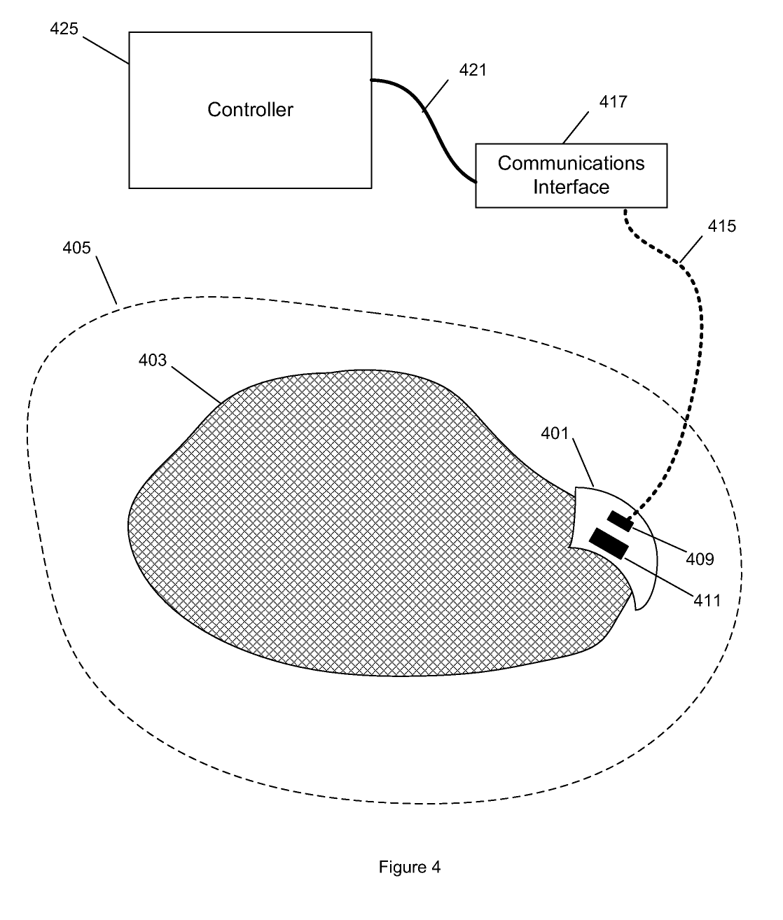

FIG. 4 shows an implantable embodiment of the above described telemetry sensor. A sensor unit 401 is placed on a tissue or organ 403, within an organ, or within a bodily cavity inside of a body 405. On the sensor unit is an antenna 409 and a power storage and regulator unit, a processor, and at least one sensing element 411. The sensor communicates with and may be energized by an RF signal 415, which is emitted from and received by an antenna and communications interface 417. The interface 417 is communicatively coupled by 421 to a controller 425 as described earlier. In one embodiment, the sensor unit 401 is placed on, or about bowel tissue 403 after performing a surgical procedure to monitor the operative site for adequate perfusion, or surgical complication such as anastomotic leak. In another embodiment the sensor can be used to detect the viability of a transplanted organ or tissue. A further embodiment would allow for the sensor to be placed in the intra-pericardial sac after cardiac procedure to detect pericardial tamponade. In another embodiment, the sensing element 401 is made in whole, or in part, of bioabsorbable/biodegradable materials including the antenna and substrate.

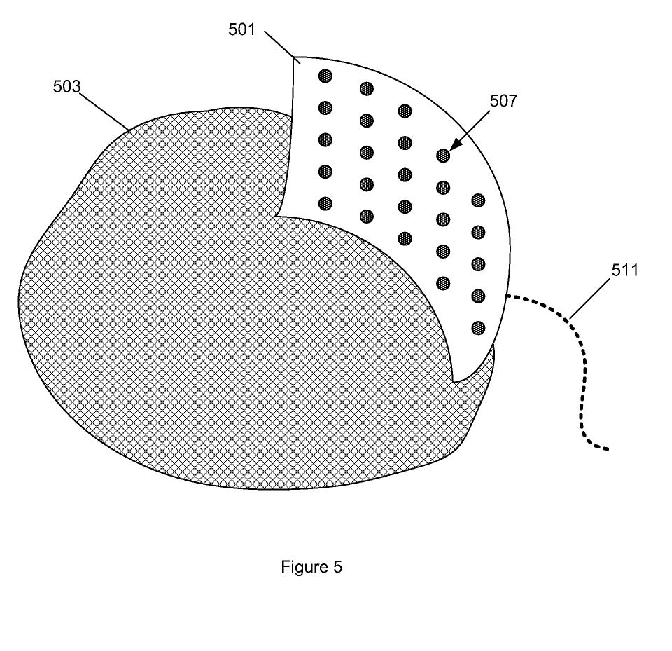

FIG. 5 shows a grid of sensing elements 507 on a sensing patch/pad/mesh 501 as described earlier. One or more sensor(s) sense one or more properties of tissue 503. Communication is wireless via RF connection 511 and the sensors are passively energized as described earlier. In one embodiment, a grid or array of one or more sensor(s) monitors perfusion in a transplanted organ or tissue.

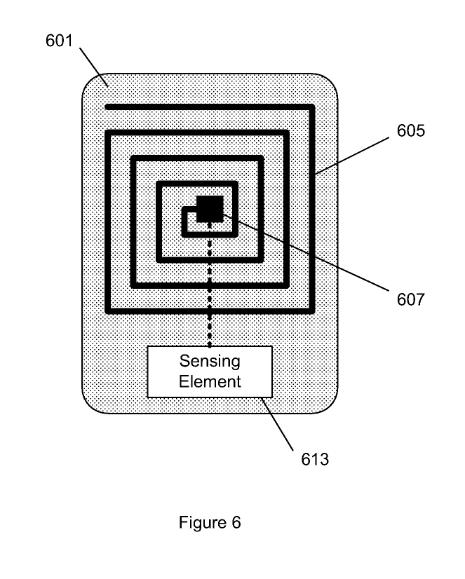

FIG. 6 shows a close up of a radio-frequency energized biosensor unit. The sensor unit is integrated into a patch or pad 601. The patch may be self adhesive, disposable, and flexible. The antenna 605 may be constructed of a conductive, biocompatible, and potentially bioabsorbable material. In one embodiment, antenna 605 may also be constructed of a bioabsorbable or biologically inert conductive ink on a bioabsorbable substrate. In another embodiment, the antenna may be constructed of conductive biocompatible gel or fluid contained in bioabsorbable tubular structures or other guides. The antenna 605 receives RF energy and uses them to generate power which is stored and regulated by controller 607. Controller 607 is coupled to antenna 605, and contains an RFID or similar transponder chip, a power regulator, and a power storage device such as a capacitor or battery. Sensing element 613 is powered by the power supply of 607. The sensing element may contain a processor, analog and digital input and outputs, a signal processor, filters, light emitters and receivers, amplifiers, temperature sensors, strain gauges, accelerometers, tilt sensors, electrodes, or other elements that would allow for sensing a physiological/biomechanical property and transmitting the data back via antenna 605. The sensing element 613 and the controller 607 may be one single integrated unit or made up of a plethora of coupled subcomponents.

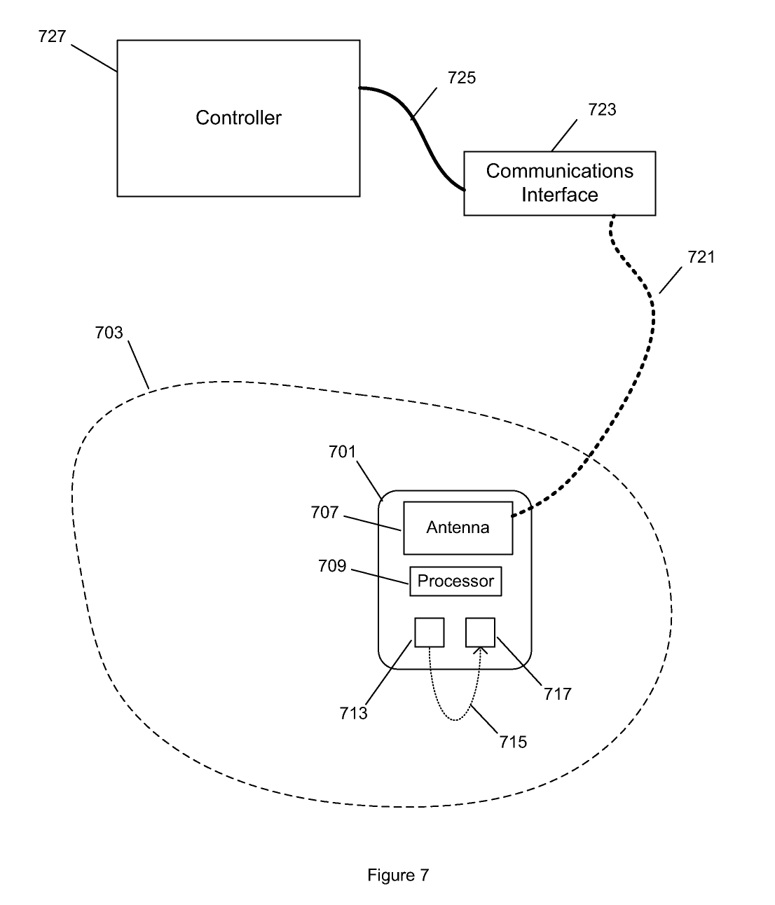

FIG. 7 shows one embodiment of the sensor for measuring oxygenation and or pulse via optical sensors. Sensing patch or pad 701 is placed on skin or other tissue 703. Antenna 707 receives power which is stored and regulated. The processor 709 commands light source 713 to transmit light. Light source 715 may be of one, two, or more wavelengths. In one embodiment, infrared and red light are transmitted sequentially as known to those skilled in the art of pulse oximetry. Light 715 is transmitted through or reflected off of tissue 703, and is detected by light sensor 717. Light sensor 717 communicates information about the respective light intensities for each wavelength to processor 709 for computation. Processor 709 transmits data via antenna 707 using techniques know to those skilled in the art of RFID. Transmissions 721 to and from antenna 707 are received by and transmitted from the communications interface 723 respectively. The communications interface 723 is coupled 725 to a controller 727 which may be a computer, stand-alone monitor, or integrated into the same device as described earlier.

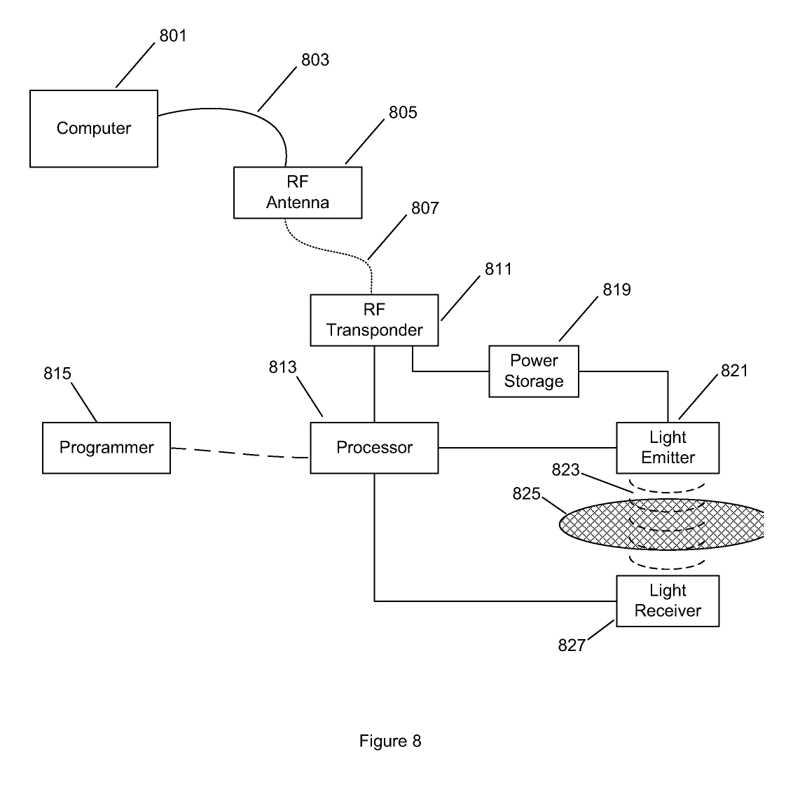

FIG. 8 shows a flowchart of one embodiment of the system described in the previous paragraph. Computer 801 is communicatively coupled via 803 to antenna device 805. Antenna 805 transmits RF signals 807 to transponder/sensor 811. In the transponder unit is a microprocessor or microcontroller 813 which is programmed either via a wired connection or wirelessly 815. Also contained in the transponder is a power storage and regulator unit 819. A processor 813 controls light emitters 821 which are powered by the energized storage unit 819. A light source 823 emits a plethora of wavelengths of light that travel through or reflect off of tissue 825 before being received by a light sensor 827 which relays data back to the processor 813. In one embodiment, the light source 823 emits red and infrared light for oximetry based measurement techniques as know by those skilled in the art. The processor 813 relays data to the transponder which is transmitted over RF signal 807 to antenna 805 which is relayed back to the computer or controller 801.

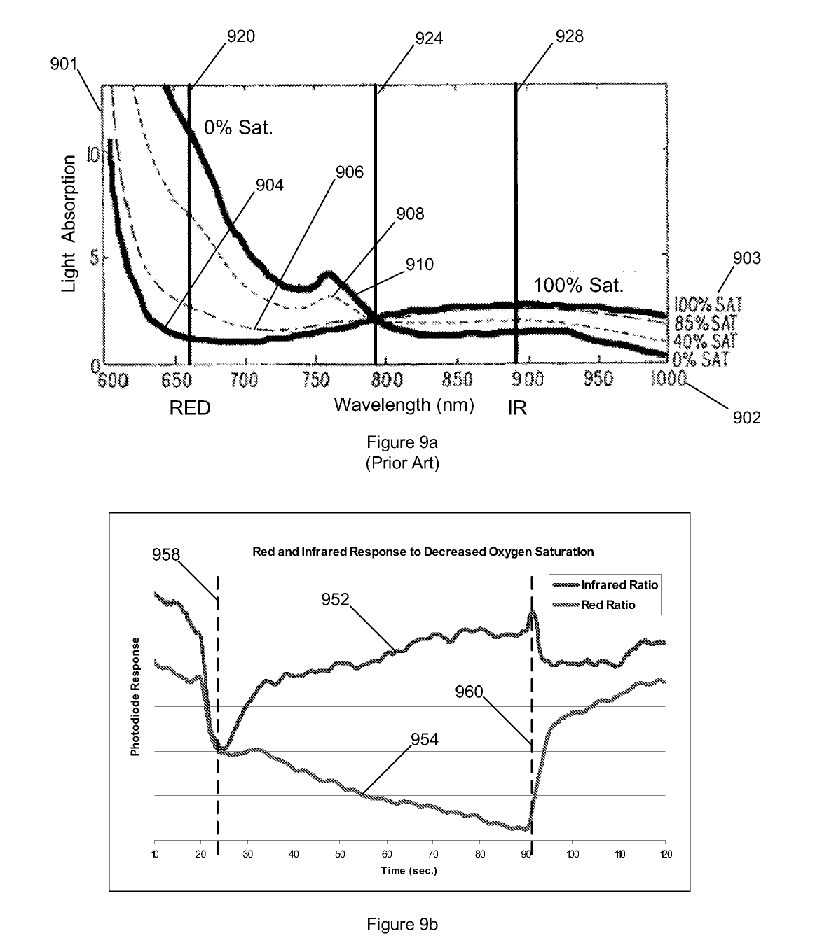

FIG. 9a illustrates the fundaments of oximetry based sensing, which are known to those skilled in the art. Light absorption 901 varies as a function of wavelength 902 and blood oxygen saturation 903. At a minimum, red light 920 at around 660 nm and infrared light 928 at around 880 nm are emitted into the tissue. Light at the isobestic wavelength 924 of about 780 nm may be used as well to increase the robustness of measurements. Further wavelengths of light may be used to increase robustness and accuracy.

FIG. 9b shows an experiment that verifies the increased attenuation of red light 954 and decreased attenuation of infrared light 952 as blood oxygen saturation is decreased starting at time 958 and restored at time 960. To sense relative changes in a patient's blood oxygen saturation, in some embodiments it is only necessary to monitor trends in this change in attenuation and not correlation to standard SpO2 values as in FIG. 9a.

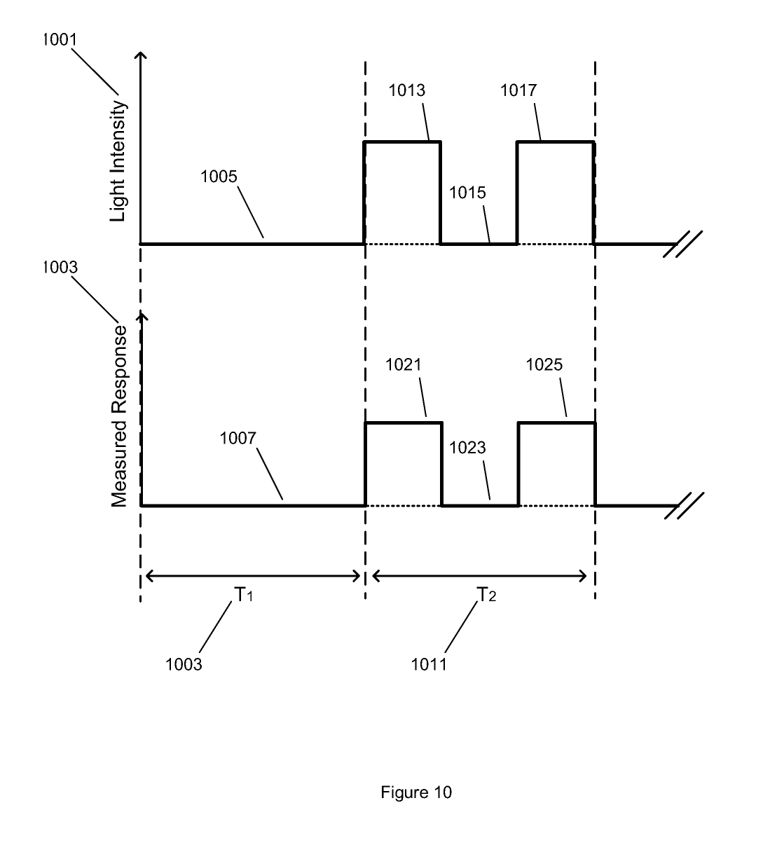

FIG. 10 shows a timing diagram for one embodiment for a simple RF energized oxygenation sensor. Representative emitted light intensity 1001 and received light intensity 1003 are plotted with respect to time. During interval 1003, the RF signal is energizing the power storage unit, and there is no light emission from the sensor. After charging is complete, light is emitted and received sequentially during time period 1011. In one embodiment, the light emissions 1013, 1015, and 1017 correspond to red light, no emitted light, and infrared light, respectively. The corresponding received light intensities are 1021, 1023, and 1025, respectively. In this embodiment, two wavelengths are transmitted; in general, one or more wavelengths can be emitted and received. If optical filters, frequency modulation, or other techniques know to those in the art are used, multiple wavelengths of light may be used simultaneously. If pulse, and not oxygenation, is being detected, it may be possible to use ambient light transmitted through tissue in place of emitted light.

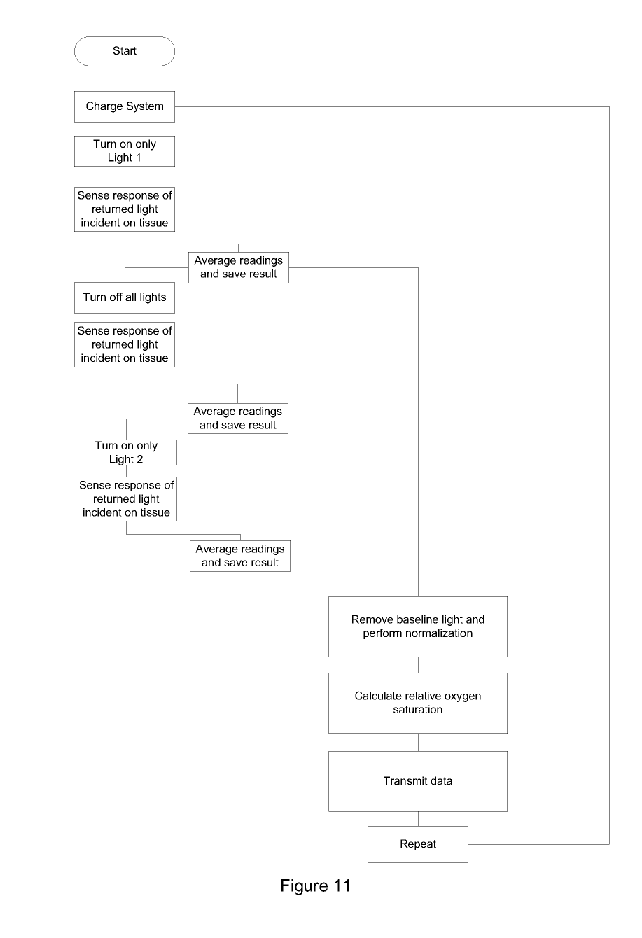

FIG. 11 shows a flow chart for the oxygenation sensor described above. After starting the system by querying the sensor, the sensor is energized by the RF signal. After the stored electrical charge is sufficient to power the sensor, red light is emitted and the signal is sensed. The same is done for the baseline of no light transmission and infrared light as described above. The baseline is subtracted out and any tissue-dependent normalization is taken into account. Oxygenation and/or pulse related information is calculated and transmitted from the sensor and the cycle repeats. Similar workflows can be implemented in other embodiments using other sensing modalities as described earlier.

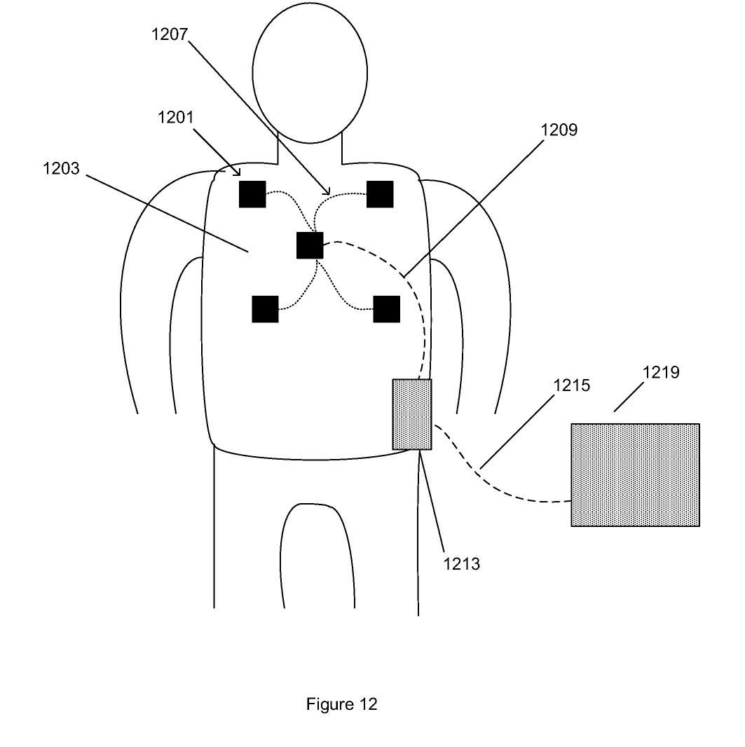

FIG. 12 shows a further embodiment of a sensing patch. The sensors are designed to monitor electrical activity and can be used for ECG monitoring. Two, three, five, or other numbers of sensing pads or patches with electrodes 1201 are placed on the skin of the chest 1203. The pads may be communicatively coupled to each other by 1207 which may be a wired or wireless connection, and may contain one or more RF telemetry devices. At least one device is energized by and communicates using the RF signal 1209. The main RF transmitter/receiver 1213 may be an external device. In one embodiment, it is a portable, wearable device. The RF transmitter/receiver 1213 may be communicatively coupled via 1215 with a base station, monitor, or other device 1219 including those described earlier. In another embodiment, similar sensors may be used to monitor EMG or other electrical activity in the body. In one embodiment, two ECG or similar electrodes are placed on opposing sides of a single dermal patch that can be worn by the patient.

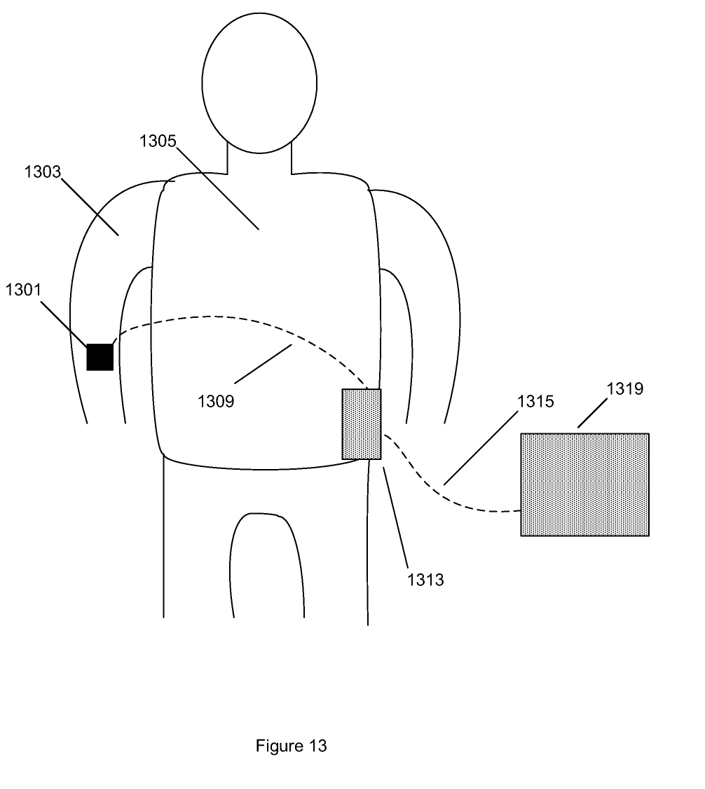

FIG. 13 shows a further embodiment comprising a wearable sensor 1301 that may take the form of dermal patch, armband, a watch, a fashion accessory, a article of clothing, or other wearable device 1301 where the sensor contains an accelerometer, tilt sensor, or other motion sensor. The sensor 1301 is placed on an extremity 1303 or on the main body 1305 itself to detect significant patient motion. The sensors are energized by and communicate with RF signal 1309. In one embodiment, the main RF transmitter/receiver 1313 is a portable, wearable device. The RF transmitter/receiver 1313 is communicatively coupled via 1315 to a base station, monitor, or other device 1319 including those described earlier. The sensor can be used for detecting motion; this can be applied to detecting a patient's attempts to get out of bed, make large motions, or remove restraints. It can also be used to alert when convulsive activity is present and to ensure that respiratory and/or cardiac motion present. A further use is for monitoring an infant for abusive or dangerous treatment.

In another embodiment, sensor 1301 represents an oxygenation and/or pulse sensor. The sensor 1301 is energized by and transmits data via RF signal 1309 to a portable monitor 1313. In this embodiment, the sensor 1301 can be used as an exercise monitor and portable monitor 1313 can be used to record and display pulse and other information to the user in real time. In one embodiment, portable monitor 1313 is integrated into or part of an add-on module to standard portable electronics including, but not limited to, mobile phones, pagers, portable computers, portable music players, or belts, clothing, jewelry, or watches. A coupling 1315 may be used to download data to a base unit, central monitoring station, or computer 1319. Coupling 1315 may be through a wired connection, through a short range wireless connection, through standard wireless internet or cellular connection, or through another network. A similar embodiment uses an independent monitor 1313 for monitoring pulse rate and other physiologic parameters. One application of such an embodiment is in military or other health care situations where a soldier or patient wears a sensing dermal patch, and a medic or healthcare professional carries a portable monitor. Alternatively the medic or healthcare provider is located at a central monitoring station, thus allowing the medical personnel or supervisors to remotely monitor the subject.

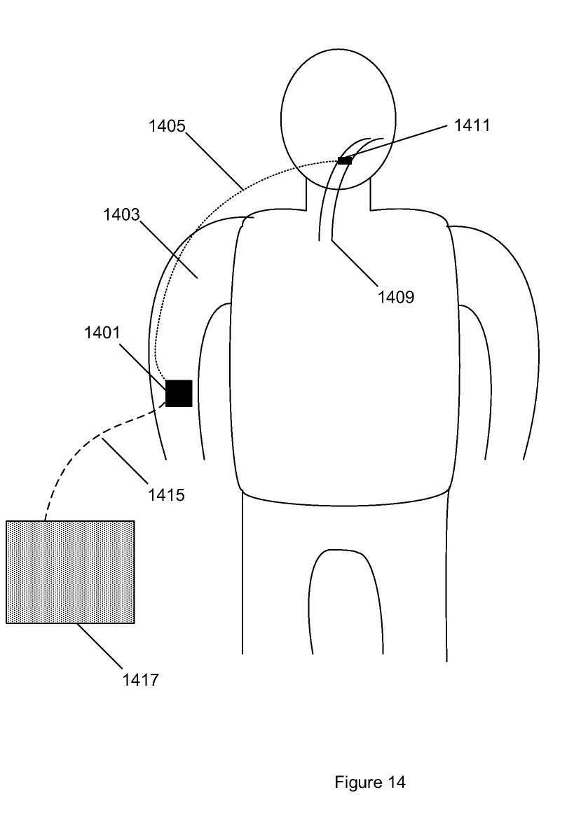

FIG. 14 shows one embodiment of a sensor 1401 incorporated into a watch, a pendant, an article of clothing, a fashion accessory, a dermal patch, an armband, or other device placed on extremity 1403 and communicates via RF signal 1405. Endotracheal tubes, IV catheters, thorocostomy tubes, urinary catheters, drainage catheters, naso-/oro-gastric tubes, percutaneous feeding tubes, electrodes, wires, and other devices 1409 have embedded RFID or similar tags 1411. When the tag 1411 comes within a specified distance of the sensor 1401, a signal is sent via 1415 to a controller, monitor, or other device 1417. An alert or other action will take place if the sensor becomes too close to the tag in the tube or other device to warn when a patient may be attempting to dislodge the device.

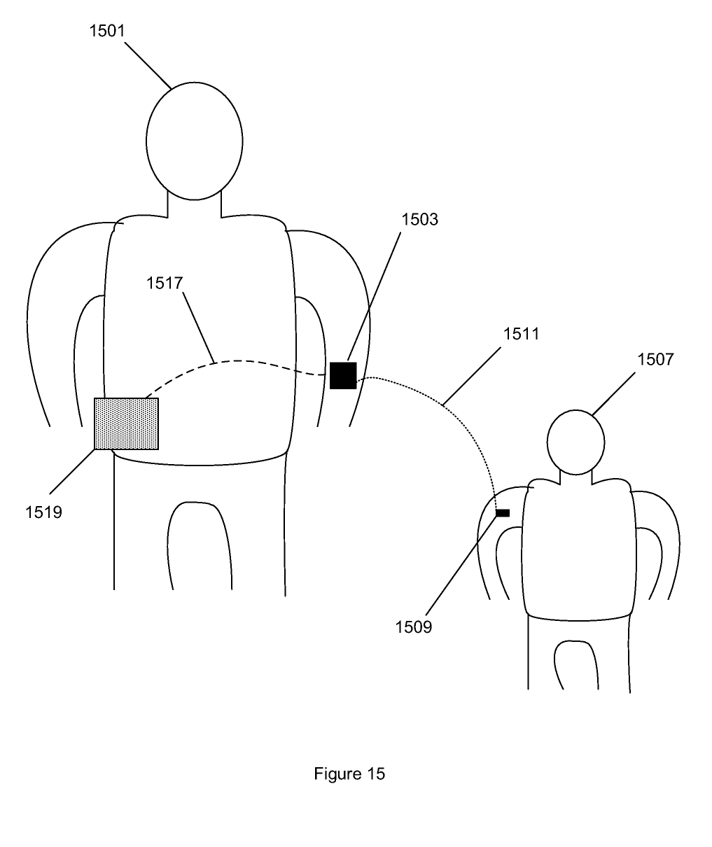

FIG. 15 illustrates an embodiment in which guardian 1501 wears or carries RFID reader or similar 1503. RFID reader or similar 1503 can be integrated into a watch, an armband, a pendant, a portable computer, a mobile phone, a portable music player, a pager, a portable gaming system, an article of clothing, a fashion accessory or other wearable accessory or portable electronic device. Child/infant or other subject 1507 wears RFID tag 1509. RFID tag 1509 can be integrated into an armband, dermal patch, piece of jewelry, article of clothing, a watch, a pendant, a portable computer, a mobile phone, a portable music player, a pager, a portable gaming system, an article of clothing, a fashion accessory, or other accessory. In another embodiment the RFID tag 1509 can be ingested, implanted or injected into the subject's body. RFID reader 1503 is communicatively coupled to RFID tag 1509 through RF signal 1511. In this embodiment RFID reader 1503 would generate a signal to alert the guardian that the infant/child has moved outside of a preset proximity. In another embodiment 1509 can be a RFEB monitoring physiologic/biomechanical properties of subject 1507. In a further embodiment the RFID reader 1503 communicates through 1517 to a communications interface 1519. This would allow for external monitoring/recording. In an alternative embodiment RFID reader 1503 is incorporated into the bed, crib, or other location. This embodiment can be used to determine if an infant tries to get out of his/her crib, if an elderly or hospital care patient tries to get out of bed, or other application where it is important to determine if the subject is within or outside of a specific range.

What is claimed is:

1. A system for monitoring infants comprising: a remotely energized sensor, a transmitter, a receiver, and a monitor; wherein the sensor detects at least one of a physiologic and biomechanical parameter of an infant or child; wherein the sensor and transmitter receive power through radio frequency (RF) energy; wherein the transmitter communicates wirelessly with a receiver in a nearby base station.

2. The system of claim 1, wherein the sensor and transmitter is integrated into a fixture comprising at least one of a dermal patch, a diaper, or an article of clothing.

3. The fixture in claim 2, wherein the fixture is disposable.

4. The system of claim 1, wherein the sensing modalities include at least one of an optical sensor, an oximetry sensor, an electrical sensor, a chemical sensor, a mechanical sensor, a MEMS sensor, a nano sensor, a biochemical sensor, an acoustic sensor, an immunologic sensor, a fluidic sensor, and a ‘lab-on-a-chip’ type sensor.

5. The system of claim 4, wherein the sensor detects at least one of general health, blood oxygen saturation, blood glucose levels, electrical activity including electrocardiogram (ECG) and electromyography (EMG), pulse presence, pulse rate and character, respiratory motion, respiratory rate, temperature, pH, chemical composition, and body motion including acceleration and mechanical shock.

6. The system of claim 1, wherein the base station takes the form of, is incorporated into, or is mechanically coupled to at least one of a toy, a mobile, a bed, a crib, a stroller, a bedding material.

7. The system of claim 1, wherein the monitor is integrated into the base station.

8. The system of claim 1, wherein a remote monitor, and optionally a data recorder, are communicatively coupled to the base station; wherein the remote monitor takes the form of at least one of a dedicated baby monitor, an alarm, a watch, an armband, a pendant, a portable computer, a mobile phone, a portable music player, a pager, a television, a remotely accessible web site, and a software interface.

9. The system of claim 1, wherein an alert is generated responsive to a condition detected by the sensor.

10. The system of claim 1, wherein the system detects early signs of sudden infant death syndrome (SIDS).

11. The system of claim 20, wherein the sensing modality is based on at least one of oximetry, infant body motion, and electrical activity.

12. The system of claim 1, wherein the system detects of fever.

13. The system of claim 1, wherein the system detects convulsive activity.

14. A system comprising: a remotely energized implantable sensor for generating a signal indicative of at least one of a physiologic and biomechanical property of a patient, a transmitter for sending the signal, a receiver unit for receiving the signal, and a processor configured to produce an output responsive to the signal.

15. The system of claim 14, wherein the sensing modalities include at least one of an optical sensor, an oximetry sensor, an electrical sensor, a chemical sensor, a mechanical sensor, a MEMS sensor, a nano sensor, a biochemical sensor, an acoustic sensor, an immunologic sensor, a fluidic sensor, and a ‘lab-on-a-chip’ type sensor.

16. The system of claim 15, wherein the sensors detect at least one of surgical procedure failure, general health, blood oxygen saturation, blood glucose levels, inflammation, electrical activity including electrocardiogram (ECG) and electromyography (EMG), pulse presence, pulse rate and character, respiratory motion, respiratory rate, temperature, pH, chemical composition, and body motion including acceleration and mechanical shock.

17. The system of claim 14, wherein the sensor and transmitter form an integrated sensing device that communicates wirelessly with a receiver unit.

18. The system of claim 17, wherein the receiver unit attaches to or is integrated into at least one of an article of clothing, a wearable device, a bed, a bedding material, a stretcher, a backboard, and an operating room table.

19. The system of claim 14, wherein the receiver unit is a portable monitoring device.

20. The system of claim 14, wherein the receiver is communicatively coupled to a remote monitoring station.

21. The system of claim 14, wherein the sensor is powered wirelessly by a radio frequency source.

22. The system of claim 14, wherein the sensing device is partially or fully absorbable into the body.

23. The system of claim 14, wherein the antenna of the sensing device is partially or fully absorbable into the body.

24. The system of claim 14, wherein the antenna is comprised of a bioabsorbable or biologically inert conductive ink printed on a bioabsorbable or biologically inert substrate.

25. The system of claim 14, wherein the antenna is comprised of conductive biocompatible gel or fluid contained in bioabsorbable tubular structures or guides.

26. The system of claim 14, wherein the system monitors and optionally records the condition at an operative site.

27. The system of claim 14, wherein the system monitors and optionally records the condition of a transplanted organ.

28. The system of claim 14, wherein the system monitors and optionally records at least one of a physiologic and biomechanical condition within at least one of an intra-cranial, intra-thecal, intra-ocular, intra-otic, intra-nasal, intra-sinusoidal, intra-pharyngeal, intra-laryngeal, intra-esophageal, intra-tracheal, intra-thoracic, intra-bronchial, intra-pericardial, intra-cardiac, intra-vascular, intra-abdominal, intra-gastric, intra-cholecystic, intra-enteric, intra-colonic, intra-rectal, intra-cystic, intra-ureteral, intra-uterine, intra-vaginal, and intra-scrotal space.

29. The system of claim 14, wherein the system monitors and optionally records at least one of a physiologic and biomechanical condition within at least one of a vascular, a cardiac, a cerebral, a pulmonic, a hepatic, a pancreatic, a renal, an adrenal, a lienal, an ovarian, a testicular, a penal, a muscular, an osseous, and a dermal tissue.

30. A system for detecting adverse patient activity comprising: a remotely energized sensor, a transmitter, a receiver, and a monitor; wherein the sensor detects an adverse patient activity; wherein the sensor and transmitter receive power through radio frequency (RF) energy; wherein the transmitter communicates wirelessly with a receiver in a nearby base station.

31. The system of claim 30, wherein the sensor and transmitter is integrated into a fixture comprising at least one of a dermal patch, an armband, or a watch, a pendant, an article of clothing, and a fashion accessory.

32. The fixture in claim 30, wherein the fixture is disposable.

33. The system of claim 30, wherein the transmitter and receiver are configured for the detection of patient motion.

34. The system of claim 30, wherein the patient motion is an attempt to dislodge indwelling or externally placed medical equipment from said patient.

35. The system of claim 34, wherein the transmitter is incorporated into at least one of an endotracheal tube, an IV catheter, a needle, a thorocostomy tube, a urinary catheter, a drainage device, a nasogastric tube, an orogastric tube, a percutaneous feeding tube, a breathing apparatus, an electrode, and a wire.

36. The system of claim 30 wherein the patient motion is an attempt by said patient to climb out of bed or to remove restraints.

37. The system of claim 30, wherein the monitor generates an alert when the transmitter enters or leaves the proximity of the receiver.
